(19) United States
(12) Patent Application Publication
(10) Pub. No.: US 2006/0189918 A1
(43) Pub. Date: Aug. 24, 2006

(54) Laparoscopic port hernia device

(76) Inventor: Stephen George Edward Barker, Surrey (GB)

Correspondence Address: NIXON & VANDERHYE, PC, 901 NORTH GLEBE ROAD, 11TH FLOOR, ARLINGTON, VA, 22203, US

(21) Appl. No.: 10/542,030

(22) PCT Filed: Jan. 12, 2004
(86) PCT No.: PCT/GB04/00129

§ 371 Date: Aug. 2, 2005

(30) Foreign Application Priority Data
Jan. 14, 2003 (GB) 0300786.1

(51) Int. Cl. A61F 13/20

(52) U.S. Cl. 604/13; 604/15

(57) ABSTRACT

An applicator assembly for use in applying a sheet of surgical material through an opening to bridge the remote internal termination of the opening has a deployment sleeve (1), a plunger (2) for location within the sleeve (1) and a sheet of surgical material (3) which can be folded or collapsed to a conical form as shown for location at and within the distal end of the deployment sleeve. An actuating means (4) comprising a suture is provided operative to unfold or erect the sheet (3) following expulsion from the distal end of the deployment sleeve (1) through longitudinal movement of the plunger (2). The sheet (3) includes radial ribs (5) connected along their length to the sheet except at an inner region (5a) defined by a hinge (5b) where the ribs are free and joined at the ends to a collar (5c) through a further hinge connection.

This invention relates to a laparoscopic surgical material and an applicator primarily intended for internal closure of an incision to prevent formation of a port-site hernia following a surgical procedure, or operation. Secondarily, prevention of formation of internal organ adhesion at the operation site may be helped.

Laparoscopic surgery is used increasingly and requires the provision of one or more “tunnels” through all layers of the body wall, forming ports through which a surgical procedure is effected remotely using various instruments, a telescope or camera and a light source. For a laparoscopic cholecystectomy, for example, three such ports are used typically, with 0.5 to 1 cm incisions down through the layers of abdominal wall, with one port very often through the umbilicus. Typically each port is closed, following the procedure, with outer skin stitches only, thus leaving the deeper layers to heal themselves. This small incision has the potential, later, to become an incisional or port hernia. Internally, the unrepaired port opening can allow, potentially, a bowel portion to herniate through or may offer an adhesion point for bowel, with the potential then for bowel obstruction.

It is a primary objective of this invention to provide a surgical material and applicator for use in the closure of port incisions, such as those following laparoscopic procedures and which is simple to use through the same laparoscopic port lumen and which has a minimum of components.

Another object is to provide a collapsed device structure which may be readily passed down the port and then opened out internally, located into and across the opening.

According to one aspect of this invention there is provided an applicator assembly for use in applying a sheet of surgical material through an opening to bridge the remote internal termination of the opening, the assembly comprising;
a) a deployment sleeve;
b) a plunger for location within the sleeve, to extend from the proximal to the distal end thereof;
c) a sheet of surgical material which can be folded, or collapsed, for location at and within the distal end of the deployment sleeve;
d) an actuating means operative to unfold or erect the sheet following expulsion from the distal end of the deployment sleeve through longitudinal movement of the plunger and, optionally,
e) a further means such as a suture, operative to apply a pull force to the sheet following deployment.

In this arrangement, and according to the invention, the sheet material is encased and protected within the deployment sleeve during the manipulation procedure to position the sleeve at the termination of the opening after which the sheet is expelled by the plunger to be erected to close behind and over the internal area of the opening.

The sheet of surgical material will generally be of a known surgically compatible mesh such as polypropylene, most likely including a PTFE or similar non-stick material on one surface, that is the innermost facing surface. The entire part however, may be wholly of PTFE, for example, “textured on the surface away from the bowel. The material is normally a flat flexible sheet, for example circular, and may include radial ribs forming more rigid but resilient arms through which, when the sheet is forced to a collapsed or folded configuration, the sheet Is caused to open out to restore the flat form. This action may be likened to the opening of an umbrella and a feature is that the opening can be effected through the properties of the sheet itself not requiring additional mechanical components. As an alternative, or in addition, opening is effected through a suture which pulls the sheet upward towards the applicator. The central part of this arrangement of arms, may locate within the internal defect to aid closure.

In an embodiment, the ribs have preformed fold creases to facilitate collapse to a predetermined configuration. Alternatively, the ribs may have a “memory” acting to restore the sheet to a flat form. Following deployment of the sheet the opening thereof may be effected or assisted by a separate actuating means which may comprise a suture thread, with or without an attached suture needle, extending through the plunger from the proximal to the distal end of the sleeve and connecting with the sheet. The suture is arranged so that a pulling force applied thereto opens the sheet. The suture may then be removed, or severed, or broken. In this embodiment, the suture may be used for applying traction to position the sheet and to close the wound using the attached needle.

According to another aspect of this invention, there is provided a sheet of surgical material including ribs or radially extending formations which may be resiliently flexed and which, on restoration, extend the sheet from a folded, pleated or crumpled form to a flat and self-supporting form.

According to yet another aspect of this invention there is provided a sheet of surgical material including radial ribs each rib being hinged to allow outer parts of the sheet to be folded inwards into a conical shape, the sheet being extended to a flat form by an actuating means. The sheet in these embodiments thus opens in the manner of an umbrella. The arms may be constructed of a biodegradable material, this being preferred but not essential.

To explain the features of this invention further reference is now made to the drawings showing an embodiment by way of an example. In the drawings:

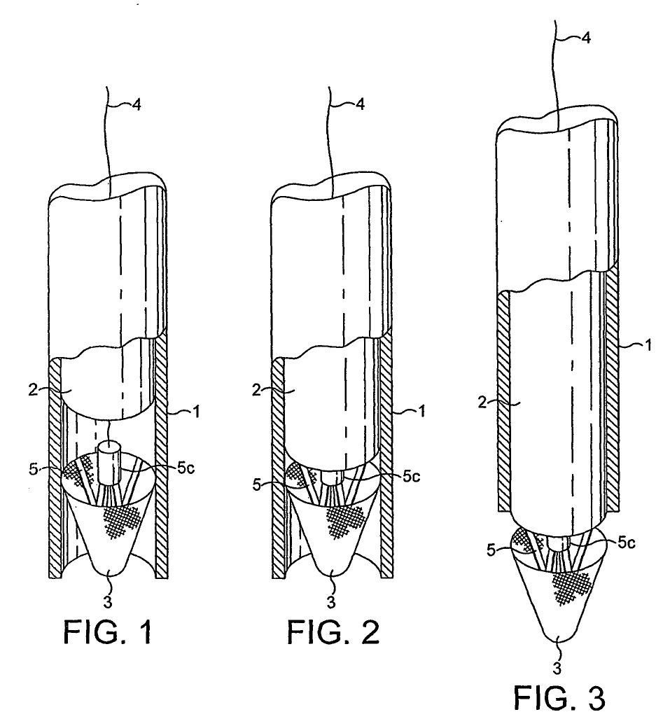

FIG. 1 shows a surgical mesh and an applicator in accordance with this invention in partial sectional view and in a first assembly stage,

FIG. 2 shows the applicator in a loaded stage,

FIG. 3 shows the applicator during initial deployment,

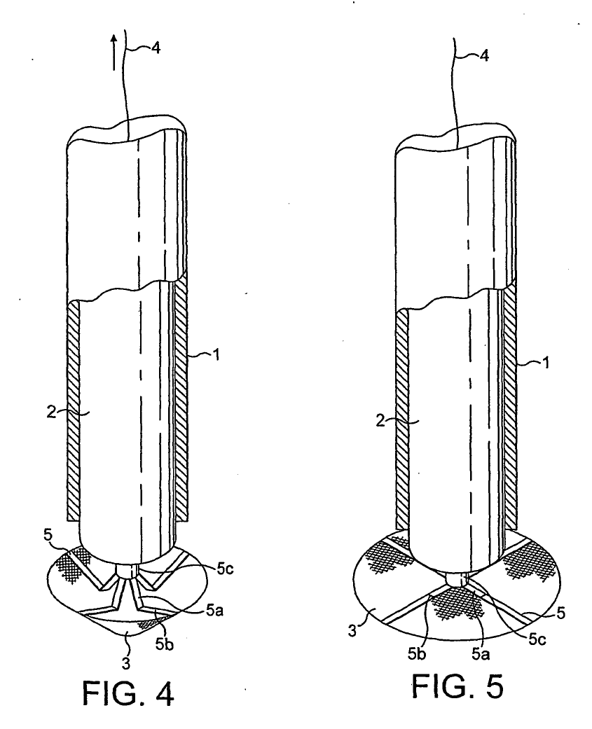

FIG. 4 shows the applicator with the sheet partially opened,

FIG. 5 shows the applicator with the sheet fully opened,

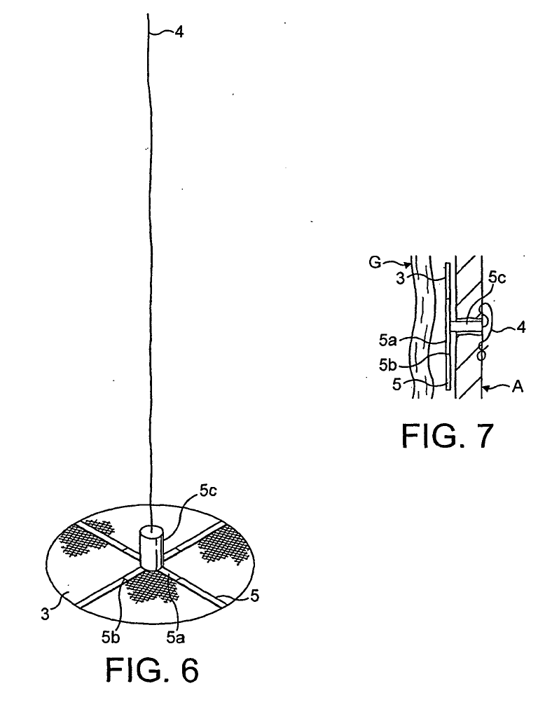

FIG. 6 shows the sheet after withdrawal of the applicator, and

FIG. 7 shows in diagrammatic form a closure.

Referring to FIG. 1 of the drawings, an applicator assembly for use in applying a sheet of surgical material through an opening to bridge the remote internal termination of the opening has a deployment sleeve 1, a plunger 2 for location within the sleeve 1 and which has a length sufficient to extend from the proximal to the distal end of the sleeve and a sheet of surgical material 3 which can be folded or collapsed to a conical form as shown for location at and within the distal end of the deployment sleeve. The material can equally well be located the other way round, that is with the apex adjacent the plunger 2. An actuating means 4 comprising a suture is provided operative to unfold or erect the sheet 3 following expulsion from the distal end of the deployment sleeve 1 through longitudinal movement of the plunger 2. The sheet 3 is conveniently circular, typically, of some 3 cm or so in diameter. The inner facing surface will include a PTFE coating or layer to inhibit adhesion of tissue, notably bowel. The outward facing surface will be of uncoated polypropylene mesh, “textured” PTFE or similar, to facilitate incorporation into the body wall tissues. Those familiar with the art will be conversant with the term “textured” PTFE as used herein.

The sheet 3 includes radial ribs 5 connected along their length to the sheet except at an inner region 5a defined by a hinge 5b where the ribs are free and joined at the ends to a collar 5c through a further hinge connection (see FIGS. 4 to 6). The hinge connection may be formed by crease lines or zones of reduced thickness or width. The ribs preferably comprise a biologically absorbable polymer material, for example Vycryl® or PDS, or they may be of a non-biodegradable material.

The suture 4 forming the actuating means connects to the centre of the sheet and passes through the collar 5c and the plunger 2 to the proximal end.

Laparoscopic ports are generally 0.5 to 1.0 cm in nominal diameter and thus sleeve 1 is appropriately dimensioned to pass down the port device. The suture may be absorbable Vycryl® or similar and pass all the way through the plunger to a needle. At the termination of the laparoscopic procedure following withdrawal of instruments the applicator assembly will be fed through the port device. The outer sleeve may, or may not, have an externally placed flange to control the length of insertion in the port and the plunger may likewise have a flange to control the position in the sleeve.

The operation is as follows:

The sleeve 1 includes the folded or collapsed mesh 3 previously located in the distal end with suture 4 fitted, and then receives the plunger 2 (FIG. 1)

The plunger 2 is then pushed down onto the collar 5c (FIG. 2)

Deployment is effected after location through a laparoscopic port device by pushing down the plunger to expel the mesh out of the end of the sleeve 1 (FIG. 3)

Suture 4 is then pulled tight causing the mesh 3 to open through the unfolding of the rib parts 5 and 5a (FIG. 4) and to present a load dissipating support mesh closing the incision.

With the mesh now fully unfolded (FIG. 5) the sleeve and plunger may be withdrawn, usually accompanied by removal of the originally placed port device, leaving the mesh 3 in position (FIG. 6).

FIG. 7 shows in a diagrammatic way the installed mesh wherein G represents the gut, as example, and A represents the abdominal wall.

In one arrangement the suture is connected to a straight needle, such that the needle can pass down the lumen of the plunger, allowing the placement system to be discarded. Alternatively, the outer sleeve and plunger could be perforated along their entire lengths, such that both could be pulled apart, down the perforation and discarded. In either case, the placement system would be removed from the port device and the port device removed secondarily. If no needle was present then all devices could be removed simultaneously, with the thread used for traction only, being tied off in to the wound by a second wound closure suture used independently. The suture could be used for directly to close the wound, if desired.

If the placement system outer sleeve had preferentially no flange, then the port device could be slid off over it, prior to mesh deployment.

In a modification of this invention and for use in similar scenarios to laparoscopic ports, e.g. at an appendicectomy, a larger sheet of mesh for example, with the inner aspect covered with PTFE (Teflon) could be placed to prevent post-operative adhesion of bowel. This mesh could have one, two or more sutures attached along its length. This arrangement would utilise the sheet which unfolds but the use of the applicator would not be essential in this case.

When placed at the end of (open) surgery, the mesh overall would help dissipate load at the wound site, helping prevent incisional hernia formation. The PTFE inner aspect would help prevent bowel or organ adhesion. The polypropylene mesh, or “textured” PTFE, outer aspect would help incorporation into the peritoneum. The attached sutures could be pulled, to tension the mesh against the inside of the abdominal wall. Attached needles could then be used to close the wound. Having different numbers of needles along a length of mesh means that the mesh could be cut to any length required. Longer mesh would necessarily be broader also.

1-22. (canceled)

23. An applicator assembly for use in applying a sheet of surgical material through an opening to bridge the remote internal termination of the opening, the assembly comprising;
a) a deployment sleeve for passing down a laparoscopic port;
b) a plunger for location within the sleeve, to extend from the proximal to the distal end thereof;
c) a sheet of surgical material which can be folded, or collapsed, for location at and within the distal end of the deployment sleeve; and
d) an actuating means operative to unfold or erect the sheet following expulsion from the distal end of the deployment sleeve through longitudinal movement of the plunger;
wherein the deployment sleeve is provided with an external flange to control the length of insertion of the deployment sleeve in the port.

24. An applicator assembly in accordance with claim 23, including a pulling means operative to apply a pull force to the sheet following deployment.

25. An applicator assembly in accordance with claim 24, wherein the pulling means is a suture.

26. An applicator assembly in accordance with claim 23, wherein the sheet material, in use, is encased and protected within the deployment sleeve during the manipulation procedure to position the sleeve at the termination of the opening after which the sheet is expelled by the plunger to be erected to close behind and over the internal area of the opening.

27. An applicator assembly in accordance with claim 23, wherein the sheet of surgical material comprises a known surgically compatible mesh such as polypropylene, preferably including a PTFE or similar non- stick material on one surface, being the innermost facing surface adjacent the applicator.

28. An applicator assembly in accordance with claim 27, wherein the entire sheet is wholly of PTFE.

29. An applicator assembly in accordance with claim 23, wherein the sheet material is normally a flat flexible sheet, preferably circular, and includes radial ribs forming more rigid but resilient arms through which, when the sheet is forced to a collapsed or folded configuration, the sheet is caused to open out to restore the flat form.

30. An applicator assembly in accordance with claim 29, wherein the opening is effected through the properties of the sheet material itself.

31. An applicator assembly in accordance with claim 23, wherein opening is effected or assisted through a suture which pulls the sheet upward towards the applicator.

32. An applicator assembly in accordance with claim 23, wherein the sheet includes ribs which have preformed fold creases to facilitate collapse to a predetermined configuration.

33. An applicator assembly in accordance with claim 23, wherein the sheet includes ribs which have a “memory” acting to restore the sheet to a flat form.

34. An applicator assembly in accordance with claim 23, wherein, in use, after deployment of the sheet the opening thereof is effected or assisted by a separate actuating means which may comprise a suture needle, with or without an attached suture, extending through the plunger from the proximal to the distal end of the sleeve and connecting with the sheet, the suture being arranged so that a pulling force applied thereto opens the sheet.

35. An applicator assembly in accordance with claim 23, wherein the plunger is provided with a flange to control the position of the plunger in the sleeve.

36. A sheet of surgical material including ribs or radially extending formations which may be resiliently flexed and which, on restoration, extend the sheet from a folded, pleated or crumpled form to a flat and self-supporting form.

37. A sheet of surgical material including radial ribs each rib being hinged to allow outer parts of the sheet to be folded inwards into a conical shape, the sheet being extended to a flat form by an actuating means.

38. A sheet of surgical material in accordance with claim 37, wherein the ribs are of a biodegradable material.

39. A combination, for use in laparoscopic surgery, of the applicator assembly of claim 23 and said laparoscopic port, wherein the deployment sleeve of the applicator assembly is appropriately dimensioned to pass down the port.

40. A combination in accordance with claim 39, wherein the laparoscopic port has a nominal diameter of 5 to 10 mm.

41. A combination in accordance with either of claims 39, additionally comprising instruments for use in the laparoscopic surgery.

42. Use of an applicator assembly in accordance with claim 23 and said laparoscopic port, for the manufacture of a combination, for use in laparoscopic surgery.
